(19) United States
(12) Patent Application Publication
(10) Pub. No.: US 2025/0090810 A1
(43) Pub. Date: Mar. 20, 2025

(54) MEDICAL DEVICE INTEGRATING AN ACCORDION TUBE-LIKE STRUCTURE

(71) Applicant: Richard Panethere, Austin, TX (US)

(72) Inventor: Richard Panethere, Austin, TX (US)

(21) Appl. No.: 18/747,702

(22) Filed: Jun. 19, 2024

Related U.S. Application Data
(60) Provisional application No. 63/539,394, filed on Sep. 20, 2023.

(51) Int. Cl. A61M 25/06; A61B 5/15

(52) U.S. Cl. CPC A61M 25/0606; A61B 5/150992

(57) ABSTRACT

Medical device for administering medications or fluids to a patient or for drawing blood from the patient including a catheter adapter. The catheter adapter includes an extension tube at one end and an accordion tube at the other end. The accordion tube extends from the catheter adapter. The accordion tube is bendable. The medical device further includes a catheter having an introducer needle needed for insertion then extracted in typical fashion. The extension tube, the catheter adapter, the accordion tube and the catheter are in fluid communication. The catheter is insertable into the vasculature of the patient. The accordion tube provides required bendability without kinking and allows the medical device to be used for drawing blood or administering medications or fluids to the patient without causing discomfort to the patient, decreasing the need for staff intervention to intravenous (IV) or IV pump, decreasing complications, maintenance and therefore decreases overall costs.

The present application claims the benefit of U.S. Provisional Application No. 63/539,394, filed Sep. 20, 2023; all of which is incorporated herein by reference.

### FIELD OF THE INVENTION

The present invention relates to medical devices. More specifically, the present invention relates to an intravenous catheter for administering fluids, blood draws, medications and other therapies directly into a peripheral vein.

### BACKGROUND OF THE INVENTION

Peripheral and central catheters are commonly used for administering medications or fluids to a patient. Optionally, the intravenous catheters are used for drawing blood. FIG. 1 shows a butterfly catheter assembly or intravenous catheter 10, in accordance with the prior art. Catheter assembly 10 includes a catheter adapter 12. Catheter adapter 12 connects to a needle hub 14. Needle hub 14 connects to an extension tube 16. Further, catheter assembly 10 includes a metal introducer needle 18 extending from catheter adapter 12. In some implementations, catheter adapter 12 includes wings 20. Wings 20 help in venipuncture and to facilitate securing catheter 18 to the patient. Catheter adapter 12 aids in insertion of catheter 18 into a vasculature of the patient. Subsequently, fluid, medication or other therapies are administered into the vasculature via introducer needle 18. The above type catheters are only meant to be used for a maximum of a few hours and are usually only used for blood draws.

Several traditional catheters for administering the medications or fluids were disclosed in the past. One such example is disclosed in a United States granted U.S. Pat. No. 11,534,591, entitled “Systems and methods for facilitating instrument delivery through a peripheral intravenous catheter” (“the '591 Patent”). The '591 Patent discloses a system for facilitating instrument delivery through a peripheral intravenous catheter may include a catheter adapter having a proximal end, a distal end, and a lumen extending there through. The catheter adapter may include a side port. The system may include an extension tube extending from the side port. The system may include a blood control valve disposed in the lumen of the catheter adapter. The system may include a peripheral intravenous catheter extending distally from the catheter adapter.

Another example is disclosed in a United States Publication No. 20230001144, entitled “Catheter Assembly Adapter, Instrument Delivery Device, and Related Methods” (“the '144 Publication”). The '144 Publication discloses a catheter assembly which includes an adapter having a distal end, a proximal end, a side port, and an adapter lumen. The catheter assembly may include a catheter hub, which may include a distal end, a proximal end, a catheter hub lumen, and another side port. The catheter assembly may include an extension tube. A distal end of the extension tube may be coupled to the other side port, and the distal end of the adapter may be coupled to a proximal end of the extension tube. A fluidic seal may be formed around a tube of an instrument advancement device when the tube is advanced into the adapter. As an example, a portion of the adapter lumen between the distal end of the adapter and the side port may include an annular protrusion configured to form the fluidic seal.

Although the above discussed disclosures are useful, they have a few problems. For instance, the catheter causes irritation or inflammation to the vein wall. The catheter leads to frequent occlusion (blockage) requiring staff to address the intravenous (IV) and IV pump to restart the infusion, infiltration (intravenous fluids moving into surrounding tissue), dislodgement, bleeding, and infection (local and systemic). Further, frequent IV failure lead to the need to replace the IV or use a more invasive central IV such as a mid-line or peripherally inserted central catheter (PICC). Additionally, the catheters require excessive staff attention and need of early IV replacement and multiple complications.

Therefore, there is a need in the art to provide an improved medical device for administering fluids, medications and other therapies without causing discomfort to the patient.

### SUMMARY

It is an object of the present subject matter to provide a medical device that avoids the drawbacks of known intravenous catheters.

It is another object of the present subject matter to provide a medical device for administering medications or fluids to the patient or for drawing blood from the patient.

In order to overcome one or more objects, the present subject matter presents a medical device for administering medications or fluids to a patient or for drawing blood from the patient. The medical device includes a catheter adapter. The catheter adapter includes an extension tube at one end and an tube at the other end, shaped in a series of folds or pleats that can expand and contract. When compressed, the tube forms tight, parallel ridges, and when extended, it reveals a uniform, wavelike pattern. These folds are evenly spaced and run consistently across the entire length of the object, creating a zigzag or fan-like appearance from the side. The overall structure can be likened to a bellows or accordion; hereafter this tube will be referred to as an “accordion tube.”

The accordion tube extends from the catheter adapter. The accordion tube is bendable and also provides additional support to the tubing. The medical device further includes a catheter having an introducer needle used for inserting the catheter then retracted in typical fashion. The extension tube, the catheter adapter, the accordion tube and the catheter are in fluid communication. The catheter is insertable into the vasculature of the patient. The accordion tube provides required bendability without kinking and allows the medical device to be used for drawing blood or administering medications and fluids to the patient without causing discomfort to the patient. The medical device decreases intravenous (IV) maintenance, staff time and need for premature IV replacement.

The advantageous feature of the present subject matter is the bendable accordion tube prevents kinking and allows flow of blood without blockage in the catheter, especially with arm and hand movements.

Another advantageous feature of the present subject matter is the accordion tube allows the catheter to be left in the patient for a longer period of time without causing discomfort to the patient. The medical device avoids a medical practitioner or medical care personnel from needing to adjust limb positions, resetting iv pump alarms, reducing staff attention and decreasing reinsertion of multiple iv's or the need of more invasive central catheters.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

A more complete understanding of the invention and its many advantages thereof will be readily appreciated as the same becomes better understood by reference to the following detailed description, when considered in connection with the accompanying drawings wherein:

FIG. 1 illustrates a catheter assembly, in accordance with the prior art;

FIG. 2 illustrates a medical device, in accordance with one embodiment of the present subject matter; and

FIG. 3 illustrates an exemplary single monolithic structure of a medical device comprising a catheter adapter, an accordion tube and a catheter, in accordance with one embodiment of the present subject matter.

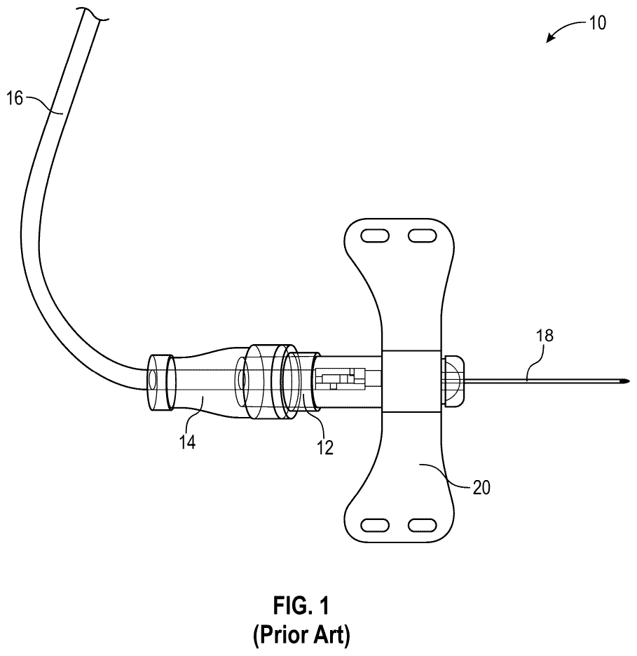

### DETAILED DESCRIPTION

The following detailed description set forth below in connection with the appended drawings is intended as a description of exemplary embodiments in which the presently disclosed subject matter may be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed medical device. However, it will be apparent to those skilled in the art that the presently disclosed subject matter may be practiced without these specific details. In some instances, well-known structures and devices are shown in functional or conceptual diagram form in order to avoid obscuring the concepts of the presently disclosed medical device.

In the present specification, an embodiment showing a singular component should not be considered limiting. Rather, the subject matter preferably encompasses other embodiments including a plurality of the same component, and vice-versa, unless explicitly stated otherwise herein. Moreover, the applicant does not intend for any term in the specification to be ascribed an uncommon or special meaning unless explicitly set forth as such. Further, the present subject matter encompasses present and future known equivalents to the known components referred to herein by way of illustration.

Although the present subject matter provides a description of a medical device, it is to be further understood that numerous changes may arise in the details of the embodiments of the medical device. It is contemplated that all such changes and additional embodiments are within the spirit and true scope of this disclosure.

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure.

The present subject matter discloses a medical device for administering medications or fluids to a patient or for drawing blood from the patient. The medical device includes a catheter adapter. The catheter adapter includes an extension tube at one end and an accordion tube at the other end. The accordion tube extends from the catheter adapter. The accordion tube is bendable. The medical device further includes a catheter having an introducer needle. The extension tube, the catheter adapter, the accordion tube and the catheter are in fluid communication. The catheter is insertable into the vasculature of the patient. The accordion tube provides required bendability without kinking, additional support and allows the medical device to be used for drawing blood or administering medications or fluids to the patient without causing discomfort to the patient and decreasing complications and reducing costs.

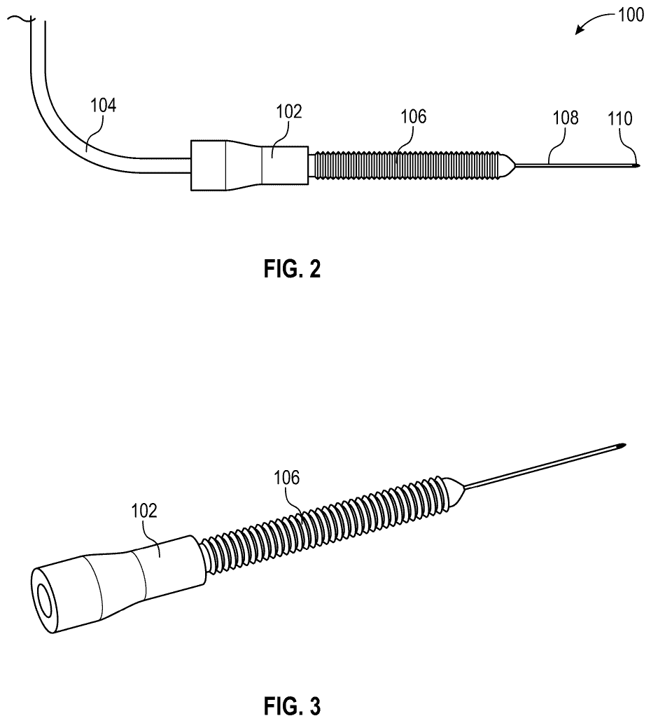

Various features and embodiments of a medical device are explained in conjunction with the description of FIGS. 2-3.

FIG. 2 shows a side view of a medical device 100, in accordance with one embodiment of the present disclosure. Medical device 100 indicates an intravenous catheter used for drawing blood or for administering medications or fluids to a patient in need. Medical device 100 includes a catheter adapter 102. Catheter adapter 102 includes an extension tube 104 at one end and an accordion tube or accordion tube-like structure 106 at the other end. In one example, extension tube 104 connects to a fluid source (not shown) such as a medicine container.

Accordion tube 106 indicates a flexible tube having a corrugated structure extending from catheter adapter 102. In one example, accordion tube 106 is made from a flexible material capable of bending and flexing while maintaining structural integrity. Medical device 100 further includes a catheter 108 having an introducer needle 110. In some instances, extension tube 104, catheter adapter 102, accordion tube 106 and catheter 108 comprise a single, monolithic structure or component. In other instances, extension tube 104, and accordion tube 106 and catheter 108 are separate devices or components that are each independently coupled to catheter adapter 102, such that extension tube 104, catheter adapter 102, accordion tube 106 and catheter 108 are in fluid communication. In some instances, catheter adapter 102, accordion tube 106 and catheter 108 comprise a single, monolithic structure or component and are in fluid communication, as shown in FIG. 3.

In the present subject matter, accordion tube 106 is capable of bending such that catheter 108 can be inserted into the vasculature of a patient. Here, catheter 108 inserts into the vasculature and accordion tube 106 provides required bendability without kinking. This allows medical device 100 to be used for drawing blood or administering medications or fluids to the patient without causing discomfort to the patient.

The presently disclosed medical device provides several advantages over prior art. For example, the medical device has an accordion tube that is bendable with increased support. As a result, the accordion tube prevents kinking from occurring especially with arm and hand movements and allows flow of blood or medicine without blockage in the catheter. The medical device can be left in the patient for a longer period of time with less need for IV maintenance, resetting of IV pump alarms or replacement of the IV.

A person skilled in the art appreciates that the medical device can come in a variety of shapes and sizes depending on the need and comfort of the user. Further, many changes in the design and placement of components may take place without deviating from the scope of the presently disclosed medical device.

In the above description, numerous specific details are set forth such as examples of some embodiments, specific components, devices, methods, in order to provide a thorough understanding of embodiments of the present subject matter. It will be apparent to a person of ordinary skill in the art that these specific details need not be employed, and should not be construed to limit the scope of the subject matter.

In the development of any actual implementation, numerous implementation-specific decisions must be made to achieve the developer's specific goals, such as compliance with system-related and business-related constraints. Such a development effort might be complex and time-consuming, but may nevertheless be a routine undertaking of design, fabrication, and manufacture for those of ordinary skill. Hence as various changes could be made in the above constructions without departing from the scope of the invention, it is intended that all matter contained in the above description or shown in the accompanying drawings shall be interpreted as illustrative and not in a limiting sense.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure.

1. (canceled)

2. A medical device for drawing blood or administering medications or fluids to a patient, said medical device comprising:
a tube, e.g. in the shape of an accordion;
an extension tube;
a catheter adapter, wherein said catheter adapter connects to said extension tube at one end and said accordion tube at another end; and
a catheter connected to said accordion tube,
wherein said accordion-shaped tube is bendable to allow said catheter to be inserted into the vasculature of a patient without kinking.

3. The medical device of claim 2, wherein said extension tube, said catheter adapter, said accordion-shaped tube, and said catheter are in fluid communication.

4. The medical device of claim 2, wherein said extension tube, said catheter adapter, said accordion-shaped tube, and said catheter comprise a single, monolithic structure.

5. The medical device of claim 2, wherein said extension tube, said accordion-shaped tube, and said catheter come as separate components and independently connect to said catheter adapter, such that said extension tube, said catheter adapter, said accordion-shaped tube, and said catheter are in fluid communication.

6. The medical device of claim 2, wherein said catheter comprises an introducer needle for inserting into the vasculature.

7. The medical device of claim 2, wherein said accordion-shaped tube comprises a corrugated structure.

8. The medical device of claim 2, wherein said accordion-shaped tube is made of a flexible material capable of bending and flexing while maintaining structural integrity.

9. The medical device of claim 2, wherein said extension tube connects to a container.

10. A medical device for drawing blood or administering medications or fluids to a patient, said medical device comprising:
an tube, e.g. in the shape of an accordion;
a catheter adapter connecting said accordion tube; and
a catheter connected to said accordion tube,
wherein said accordion tube is bendable to allow said catheter to be inserted into the vasculature of a patient without kinking.

11. The medical device of claim 10, further comprises an extension tube, wherein said extension tube connects to said catheter adapter at one end, and wherein said accordion-shaped tube connects to said catheter adapter at another end.

12. The medical device of claim 11, wherein said extension tube, said catheter adapter, said accordion-shaped tube, and said catheter are in fluid communication.

13. The medical device of claim 11, wherein said extension tube, said catheter adapter, said accordion-shaped tube, and said catheter comprise a single, monolithic structure.

14. The medical device of claim 11, wherein said extension tube, said accordion-shaped tube, and said catheter come as separate components and independently connect to said catheter adapter, such that said extension tube, said catheter adapter, said accordion-shaped tube, and said catheter are in fluid communication.

15. The medical device of claim 10, wherein said catheter comprises an introducer needle for inserting into the vasculature.

16. The medical device of claim 10, wherein said accordion-shaped tube comprises a corrugated structure.

17. The medical device of claim 10, wherein said accordion-shaped tube is made of a flexible material capable of bending and flexing while maintaining structural integrity.

18. A method of providing a medical device for drawing blood or administering medications or fluids to a patient, said method comprising the steps of:
providing a tube, e.g. in the shape of an accordion;
providing an extension tube;
providing a catheter adapter, said catheter adapter connecting said extension tube at one end and said accordion-shaped tube at another end;
providing a catheter connected to said accordion-shaped tube; and
allowing said catheter to be inserted into the vasculature of a patient without kinking while via said bendable accordion-shaped tube.

19. The method of claim 18, further comprising providing said extension tube, said catheter adapter, said accordion-shaped tube, and said catheter to be in fluid communication.

20. The method of claim 18, further comprising providing said extension tube, said catheter adapter, said accordion-shaped tube, and said catheter as a single, monolithic structure.

21. The method of claim 18, further comprising providing an introducer needle at said catheter needle for inserting into the vasculature.
